(19) United States
(12) Patent Application Publication
(10) Pub. No.: US 2005/0148033 A1
(43) Pub. Date: Jul. 7, 2005

(54) Blood compatibility testing method and device

(76) Inventors: Lori Cross, Kansas City, MO (US); Jeffrey Fry, Lansing, KS (US); Arthur Hauck, Kansas City, MO (US)

Correspondence Address: SHOOK, HARDY & BACON L.L.P., 2555 GRAND BOULEVARD, KANSAS CITY, MO, 64108-2613, US

(21) Appl. No.: 11/027,307

(22) Filed: Dec. 30, 2004

Related U.S. Application Data
(60) Provisional application No. 60/534,290, filed on Jan. 5, 2004.

(51) Int. Cl. C12Q 1/68; G01N 33/567; A61M 31/00; G01N 33/555

(52) U.S. Cl. 435/7.21; 436/520; 604/500

(57) ABSTRACT

The invention relates to a method and device for testing a blood product and a patient's blood immediately prior to transfusing the blood product to the patient. The device comprises a testing chamber within which the blood product and the patient blood are drawn and tested for compatibility. A first end of the testing chamber is coupled with an infusion needle in communication with the patient. A second end of the testing chamber is in fluid communication with the container housing the blood product. In an embodiment, blood product and patient blood are drawn into the testing chamber. One of a number of tests for determining blood compatibility is performed in the testing chamber. If the blood product and patient blood are compatible, blood product is delivered to the patient through a transfusion line bypassing the testing chamber.

### CROSS-REFERENCE TO RELATED APPLICATIONS

This application claims the benefit of U.S. Provisional Application No. 60/534,290, filed Jan. 5, 2004.

### STATEMENT REGARDING FEDERALLY SPONSORED RESEARCH OR DEVELOPMENT

Not applicable.

### TECHNICAL FIELD

The present invention relates to a method and device for ensuring compatibility of a donor blood product with the blood of a blood transfusion recipient. More specifically, the present invention relates to a method and device for testing a sample of patient blood and a sample of the donor blood product immediately prior to the transfer of the donor blood product to the patient.

### BACKGROUND OF THE INVENTION

Blood transfusions are oftentimes critical to the welfare of those undergoing medical procedures involving the loss of significant volumes of the blood. However, the dangers to a patient receiving incompatible blood are significant and have been well documented. Improvements in blood screening methods and devices have reduced the likelihood of infusing incompatible blood products. Further, the use of information technology solutions has reduced the potential for human error during the steps preceding a blood transfusion. However, despite these improvements, there remains a need for a conclusive method and device for testing donor blood against the potential recipient's blood at the last point in time prior to a transfusion that positively verifies that the donor blood product is compatible with the patient's blood.

### SUMMARY OF THE INVENTION

In some embodiments, the invention relates to a method and device for testing the compatibility between a blood product and a patient's blood immediately prior to transfusing the blood product to the patient. Thus, in one aspect, an embodiment of the present invention relates to a method for testing blood compatibility at the point of transfusion of a blood product to a patient. At least one testing chamber is provided in fluid communication with a blood product source and a patient. Blood product is drawn from the blood product source into the at least one testing chamber. Patient blood is drawn from the patient into the at least one testing chamber. The compatibility of the blood product and the patient blood is then determined. If the blood product and the patient blood are compatible, the blood product is allowed to flow to the patient through a line bypassing the at least one testing chamber.

In another aspect of the invention, an exemplary embodiment is directed to a device for testing blood compatibility at the point of transfusion of a blood product to a patient. The device includes at least one testing chamber, at least one blood product testing line, at least one patient blood testing line, and at least one line bypassing the at least one testing chamber. The at least one testing chamber is in fluid communication with a blood product source and a patient and allows the determination of the compatibility of a blood product and a patient blood that are drawn into the at least one testing chamber from the blood product source and patient. The at least one blood product testing line provides fluid communication between the blood product source and the at least one testing chamber. The at least one patient blood testing line provides fluid communication between the patient and the at least one testing chamber. The at least one line bypassing the at least one testing chamber allows the blood product to flow from the blood product source to the patient if the blood product and the patient blood are compatible.

### BRIEF DESCRIPTION OF THE DRAWINGS

The present invention is described below in detail with reference to the attached drawing figures, wherein:

FIG. 1 is a perspective view of the blood compatibility device in accordance with an embodiment of the present invention; and

FIG. 2 is a flow diagram of a method for testing the compatibility of patient blood and blood product in accordance with an embodiment of the present invention.

### DETAILED DESCRIPTION OF THE INVENTION

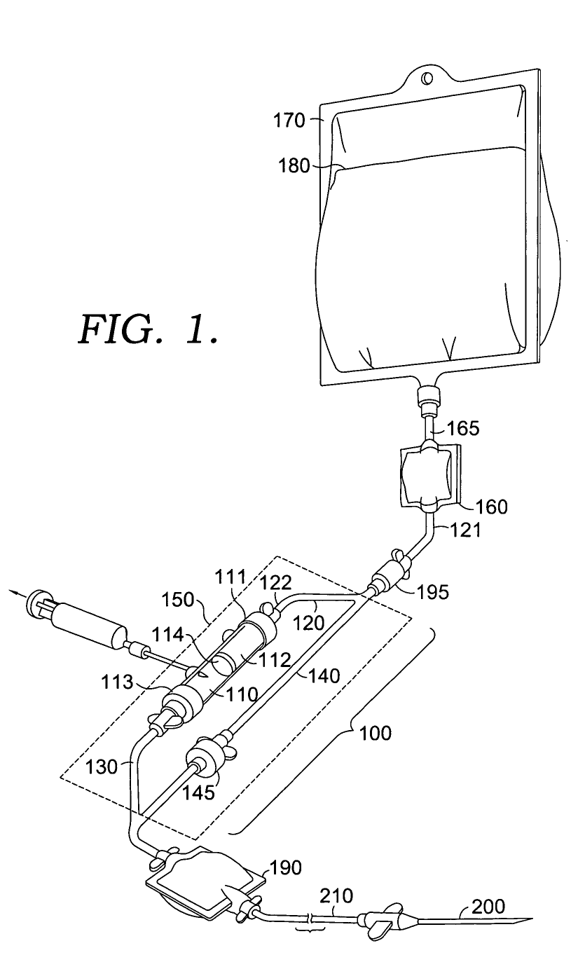

With reference to FIG. 1, a blood compatibility device 100 in accordance with an embodiment of the invention is shown. Device 100 includes a testing chamber 110, a blood product testing line 120, a patient blood testing line 130, a transfusion bypass line 140, and a housing 150. Lines 120, 130 and 140 are preferably constructed from small diameter flexible tubing known in the art for delivering blood to a patient. A first end 121 of blood product testing line 120 is in communication with a filter set 160. Filter set 160 is in fluid communication with a container 170 housing a donor blood product 180 via a line 165 extending therebetween. A second end 122 of blood product testing line 120 is coupled with testing chamber 110. A one-way check valve 195 is located along blood product testing line 120 between the first and second ends. Valve 195 only allows flow of the blood product 180 in the direction of the testing chamber 110. While the valve 195 is shown outside of housing 150, in an alternative embodiment, the valve is located within the housing.

A one-way check valve 111 is located on the end of the testing chamber 110 at which the blood product testing line 120 is in communication with the testing chamber. Valve 111 allows the flow of blood product 180 into the chamber but does not allow blood to exit the chamber. In an alternative, the one-way check valve may be located on the blood product testing line 120 leading to the testing chamber (and beyond the point at which the bypass line 140 diverges from the blood product testing line 120). The body 112 of the testing chamber 110 is transparent and is preferably made from glass or a transparent plastic cylinder. In an embodiment, the test chamber is divided into two sections by a valve 114 controllable either manually or mechanically such as by a solenoid. In this embodiment, testing chamber 110 is under sufficient vacuum pressure to draw blood from the patient and blood product 180 from the container 170 into the two respective sections of the chamber as described in greater detail below. The vacuum pressure may be created by manually drawing air from the testing chamber 110. By way of example, the chamber may have an aperture on which a syringe is attached. When the plunger is drawn, vacuum pressure is created to draw blood into the chamber.

A second one-way check valve 113 is located on the end of the testing chamber 110 opposite the blood product testing line 120. Check valve 113 is in communication with patient blood testing line 130 and only allows the flow of patient blood into the testing chamber 110. In an alternative, the check valve 113 may be independent of the testing chamber on patient blood testing line 130.

The patient blood testing line 130 extends from the testing chamber 110 to an infusion set 190. The infusion set 190 is in communication with an infusion needle 200 via a transfusion line 210.

Transfusion bypass line 140 is located in parallel with the testing chamber 110. A valve 145 is disposed along the bypass line 140. When the valve is closed, no flow is allowed through the bypass line. When the valve is open, blood product 180 is allowed to flow in the direction of the infusion set 190 and needle 200. In an alternative, valve 195 is located at the juncture of blood product line 120 and bypass line 140 and selectively allows blood product 180 through one of the blood product line 120 and bypass line 140.

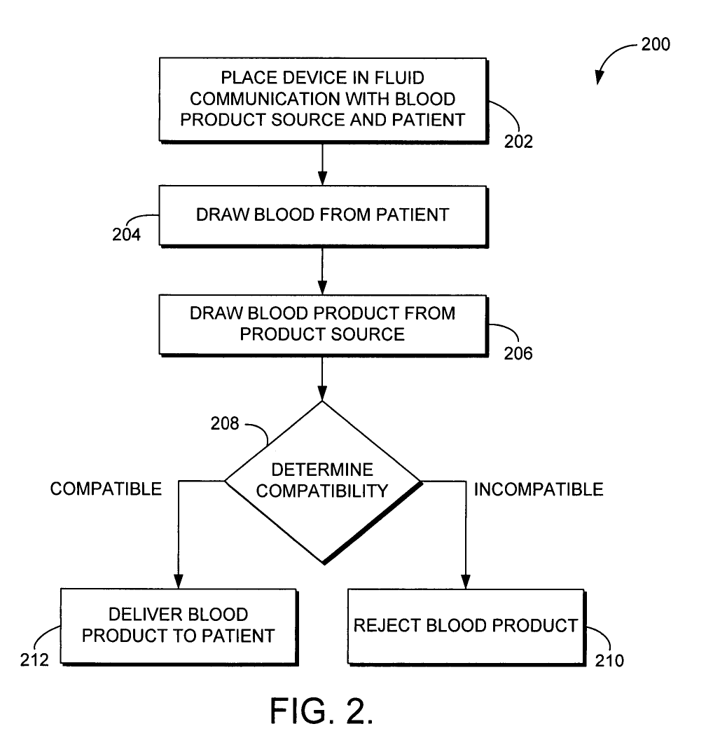

Referring now to FIG. 2, a flow diagram of an exemplary embodiment of the invention is illustrated which shows a method 200 for testing the compatibility of the blood product 180 and the patient's blood using testing device 100. The process begins at step 202 of FIG. 2 when testing device 100 is placed in fluid communication with blood product container 170 and the patient. First, container 170 containing blood product 180 is placed in communication with line 165. The infusion needle 200 is inserted in the patient. At this point, valve 145 is in the closed position, preventing any blood product 180 from flowing from container 170 to the patient.

Next, patient blood is drawn from the patient to the testing chamber 110, as shown at step 204 of FIG. 2. Valve 113 is opened and patient blood is drawn from the patient, through infusion set 190 and into the testing chamber 110. At step 206 of FIG. 2, blood product 180 is also drawn from container 170 to the testing chamber 110. Valve 111 is opened and blood product 180 is drawn from container 170, through the filter set 160 and valve 195 and into the testing chamber 110.

Patient blood and blood product 180 may or may not be mixed in the testing chamber 110. In one embodiment, the valve 114 remains closed, such that the patient blood and blood product 180 remain separate in the testing chamber 110. In another embodiment, the dividing valve 114 is opened, and the patient blood and blood product 180 are mixed with one another in the testing chamber 110.

At step 208 of FIG. 2, the compatibility of the patient blood and blood product 180 is determined. Any of a number of blood tests known in the art may be conducted to determine if the patient blood and blood product 180 are compatible with one another.

In an exemplary embodiment, the inside walls of either all or half of the testing chamber 110 are coated with AHG. Either the patient blood alone, blood product alone, or the combined blood may be visually inspected to determine if red blood cells are adhering to the walls.

In another exemplary embodiment, a major crossmatch between the patient blood serum and the donor's red blood cells (RBCs) is conducted within testing chamber 110. The patient blood is passed through a filter (not shown) to remove the red blood cells, and the remaining patient serum is drawn into the testing chamber 110. A volume of saline solution is placed in the testing chamber prior to the process of drawing blood into the chamber.

In yet another exemplary embodiment, the testing chamber 110 is removed from the system so that crossmatch testing can occur as known in the art. In an embodiment, the testing chamber is removed from the housing and the additional steps in the crossmatch process are conducted such as centrifuging or heating the blood contents.

Other examples of blood compatibility include using a liquid or solid-phase system to determine compatibility. A serologic reaction may be employed to determine patient and blood product 180 compatibility. A mechanism may be provided to mix the patient's plasma with the red cells from the blood product 180 and to visually determine if agglutination (of the blood product 180 red cells) is present. Preferably, the reaction would occur within the closed system and there would be a visual indicator, like a +/−, that would be viewable to the person performing the transfusion.

If the patient blood and blood product 180 are determined to be incompatible, the blood product 180 is rejected, as shown at step 210 of FIG. 2. If the patient blood and blood product 180 are determined to be compatible, blood product 180 is delivered to the patient, as shown at step 212 of FIG. 2. Valve 145 is opened and the blood product 180 is allowed to bypass testing chamber 110 through bypass line 140. If vacuum pressure is used to draw blood into the chamber, it is preferably discontinued at this point. Blood product 180 passes through the filter set 160, valve 195, bypass line 140, valve 145, infusion set 190 and into the patient through the needle 200.

The present invention has been described in relation to particular embodiments, which are intended in all respects to be illustrative rather than restrictive. Alternative embodiments will become apparent to those of ordinary skill in the art to which the present invention pertains without departing from its scope. Substitutions may be made and equivalents employed herein without departing from the scope of the invention as recited in the claims. For example, additional steps may be added and steps omitted without departing from the scope of the invention. It will be understood that certain features and subcombinations are of utility and may be employed without reference to other features and subcombinations. This is contemplated and within the scope of the claims.

1. A method for testing blood compatibility at the point of transfusion of a blood product to a patient, the method comprising:
providing at least one testing chamber in fluid communication with a blood product source and a patient;
drawing blood product from the blood product source into the at least one testing chamber;
drawing patient blood from the patient into the at least one testing chamber;
determining if the blood product and the patient blood are compatible; and
if the blood product and the patient blood are compatible, allowing the blood product to flow to the patient through a line bypassing the at least one testing chamber.

2. The method of claim 1, wherein the blood product and the patient blood are separated in the at least one testing chamber.

3. The method of claim 1, wherein the blood product and the patient blood are allowed to mix with one another in the at least one testing chamber.

4. The method of claim 1, wherein determining if the blood product and the patient blood are compatible comprises providing a visual indication indicative of the compatibility of the blood product and the patient blood.

5. The method of claim 1, wherein the at least one testing chamber has at least one internal wall.

6. The method of claim 5, wherein determining if the blood product and the patient blood are compatible comprises:
coating the at least one internal wall of the at least one testing chamber with AHG; and
determining if red blood cells from the blood product and the patient blood adhere to the at least one internal wall.

7. The method of claim 1, wherein drawing patient blood comprises:
filtering red blood cells from the patient blood to produce patient blood serum; and
drawing the patient blood serum into the at least one testing chamber.

8. The method of claim 7, wherein determining if the blood product and the patient blood are compatible comprises conducting crossmatch testing between the patient blood serum and the blood product.

9. The method of claim 8, wherein the at least one testing chamber is no longer in fluid communication with the blood product source and the patient during the crossmatch testing.

10. The method of claim 1, wherein vacuum pressure draws the patient blood and the blood product into the at least one testing chamber.

11. The method of claim 1, wherein the blood product is whole blood, red blood cells, blood plasma, blood platelets, or a combination thereof.

12. A device for testing blood compatibility at the point of transfusion of a blood product to a patient, the device comprising:
at least one testing chamber in fluid communication with a blood product source and a patient, wherein the at least one testing chamber is adapted to allow the determination of the compatibility of a blood product and a patient blood that are drawn into the at least one testing chamber from the blood product source and patient;
at least one blood product testing line providing fluid communication between the blood product source and the at least one testing chamber;
at least one patient blood testing line providing fluid communication between the patient and the at least one testing chamber; and
at least one line bypassing the at least one testing chamber and adapted to allow blood product to flow from the blood product source to the patient if the blood product and the patient blood are compatible.

13. The device of claim 12, wherein the at least one bypassing line is located in parallel with the at least one testing chamber.

14. The device of claim 12, wherein the blood product and the patient blood are separated in the at least one testing chamber.

15. The device of claim 12, wherein the blood product and the patient blood are allowed to mix with one another in the at least one testing chamber.

16. The device of claim 12, wherein the at least one testing chamber is divided into sections by at least one valve.

17. The device of claim 12, wherein the at least one testing chamber provides a visual indication indicative of the compatibility of the blood product and the patient blood.

18. The device of claim 12, wherein the at least one testing chamber has at least one internal wall.

19. The device of claim 18, wherein the at least one internal wall of the at least one testing chamber is coated with AHG for the purpose of determining if the blood product and the patient blood are compatible.

20. The device of claim 12, wherein the at least one patient blood testing line includes at least one filter for filtering red blood cells from the patient blood to produce patient blood serum.

21. The device of claim 20, wherein compatibility of the blood product and the patient blood may be determined by conducting crossmatch testing between the patient blood serum and the blood product in the at least one testing chamber.

22. The device of claim 21, wherein the at least one testing chamber is no longer in fluid communication with the blood product source and the patient during the crossmatch testing.

23. The device of claim 12, wherein vacuum pressure draws the blood product and the patient blood into the at least one testing chamber.

24. The device of claim 12, wherein the blood product is whole blood, red blood cells, blood plasma, blood platelets, or a combination thereof.
